(19) United States
(12) Patent Application Publication
(10) Pub. No.: US 2016/0272627 A1
(43) Pub. Date: Sep. 22, 2016

(54) POLYMORPHIC FORMS OF SUVOROXANT

(71) Applicant: DR. REDDY'S LABORATORIES LIMITED, Hyderabad, AP (IN)

(72) Inventors: Vishweshwar PEDDY, Hyderabad (IN); Deepika PATHIVADA, Hyderabad (IN); Srinivas ENUGULA, Secunderabad (IN); Arjun Kumar TUMMALA, Visakhapatnam (IN)

(21) Appl. No.: 14/442,269

(22) PCT Filed: Nov. 12, 2013
(86) PCT No.: PCT/IB2013/060080

§ 371 Date: May 12, 2015

(30) Foreign Application Priority Data
Nov. 12, 2012 (IN) 4742/CHE/2012
Dec. 19, 2012 (IN) 5322/CHE/2012

(51) Int. Cl. C07D 413/14

(52) U.S. Cl. CPC C07D 413/14

(57) ABSTRACT

Present invention relates to crystalline form of suvorexant, amorphous form of suvorexant, amorphous solid dispersion of suvorexant, processes for their preparation and pharmaceutical dosage form thereof.

Present invention relates to crystalline form of suvorexant, amorphous form of suvorexant, amorphous solid dispersion of suvorexant, processes for their preparation and pharmaceutical dosage form thereof.

### BACKGROUND

Suvorexant is chemically described as 5-chloro-2-{(5R)-5-methyl-4-[5-methyl-2-(2H-1,2,3-triazol-2-yl)benzoyl]-1,4-diazepan-1 yl}-1,3-benzoxazole or [(R)-4-(5-chloro-benzooxazol-2-yl)-7-methyl-[1,4]diazepan-1-yl]-(5-methyl-2-[1,2,3]triazol-2-yl-phenyl)-methanone or [(7R)-4-(5-chloro-1,3-benzoxazol-2-yl)-7-methyl-1,4-diazepan-1-yl][5-methyl-2-(2H-1,2,3-triazol-2-yl)phenyl]methanone. It has the structure of formula (I).

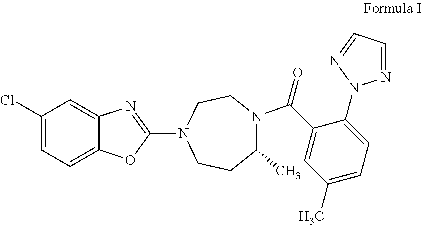 [SMILES: Cc1ccc(-n2nccn2)c(C(=O)N2CCN(c3nc4cc(Cl)ccc4o3)CC[C@H]2C)c1]

Suvorexant is an antagonist of orexin receptors and is under development for the potential oral treatment of chronic insomnia by Merck & Co. Breslin et al. U.S. Pat. No. 7,951,797 B2 discloses suvorexant or a pharmaceutically acceptable salt thereof. International Application Publication No. WO 2012/148553 A1 discloses crystalline forms of suvorexant.

Polymorphism is defined as “the ability of a substance to exist as two or more crystalline phases that have different arrangement and/or conformations of the molecule in the crystal lattice. Discovering new polymorphic forms of a pharmaceutical product may provide materials having desirable processing properties, such as ease of handling, ease of processing, storage stability, dissolution rate, ease of purification. Such properties may significantly influence the processing, shelf life, and commercial acceptance of a polymorph.

There remains a need to provide new polymorphic forms of suvorexant and processes for making the new polymorphic forms.

### BRIEF DESCRIPTION OF THE DRAWINGS

FIG. 1 is an illustration of a powder X-ray diffraction (PXRD) pattern of Form A of suvorexant.

FIG. 2 is an illustration of a powder X-ray diffraction (PXRD) pattern of Form B of suvorexant.

FIG. 3 is an illustration of a powder X-ray diffraction (PXRD) pattern of Form C of suvorexant.

FIG. 4 is an illustration of a powder X-ray diffraction (PXRD) pattern of Form D of suvorexant.

FIG. 5 is an illustration of a powder X-ray diffraction (PXRD) pattern of Form E of suvorexant.

FIG. 6 is an illustration of a powder X-ray diffraction (PXRD) pattern of amorphous form of suvorexant.

FIG. 7 is an illustration of a powder X-ray diffraction (PXRD) pattern of an amorphous form of suvorexant prepared according to Example 71.

FIG. 8 is an illustration of a powder X-ray diffraction (PXRD) pattern of an amorphous form of suvorexant prepared according to Example 74.

FIG. 9 is an illustration of a powder X-ray diffraction (PXRD) pattern of an amorphous solid dispersion of suvorexant prepared according to Example 75.

FIG. 10 is an illustration of a powder X-ray diffraction (PXRD) pattern of Form G of suvorexant.

FIG. 11 is an illustration of a powder X-ray diffraction (PXRD) pattern of Form H of suvorexant.

### SUMMARY

In an aspect, the present invention provides a crystalline form of suvorexant, designated as Form A.

In an aspect, the present invention provides a crystalline form of suvorexant, designated as Form B.

In an aspect, the present invention provides a crystalline form of suvorexant, designated as Form C.

In an aspect, the present invention provides a crystalline form of suvorexant, designated as Form D.

In an aspect, the present invention provides a crystalline form of suvorexant, designated as Form E.

In an aspect, the present invention provides a crystalline form of suvorexant, designated as Form G.

In an aspect, the present invention provides a crystalline form of suvorexant, designated as Form H.

In an aspect, the present invention provides an amorphous form of suvorexant.

In an aspect, the present invention provides a pharmaceutical composition comprising an amorphous form of suvorexant along with one or more pharmaceutically acceptable carriers, excipients, or diluents.

In an aspect, the present invention provides an amorphous solid dispersion of suvorexant together with one or more pharmaceutically acceptable carriers, or excipients.

In an aspect, the present invention provides an amorphous solid dispersion of suvorexant together with povidone.

In an aspect, the present invention provides an amorphous solid dispersion of suvorexant together with hydroxypropyl methyl cellulose.

In an aspect, the present invention also provides pharmaceutical formulations comprising amorphous solid dispersions of suvorexant together with one or more pharmaceutically acceptable carriers, excipients, or diluents.

### DETAIL DESCRIPTION

The term “about” when used in the present invention preceding a number and referring to it, is meant to designate any value which lies within the range of ±10%, preferably within a range of ±5%, more preferably within a range of ±2%, still more preferably within a range of ±1% of its value. For example “about 10” should be construed as meaning within the range of 9 to 11, preferably within the range of 9.5 to 10.5, more preferably within the range of 9.8 to 10.2, and still more preferably within the range of 9.9 to 10.1.

Room temperature as used herein refers to ‘the temperatures of the thing close to or same as that of the space, e.g., the room or fume hood, in which the thing is located. Typically, room temperature is from about 20° C. to about 30° C., or about 22° C. to about 27° C., or about 25° C.

A process or step may be referred to herein as being carried out “overnight”. This refers to a time interval, e.g., for the process or step, that spans the time during the night, when that process or step may not be actively observed. This time interval is from about 8 to about 18 hours, typically about 16 hours.

Unless indicated, the solid state forms of the present invention may be dried. Drying may be carried out, for example, at elevated temperature with or without reduced pressure.

Drying may be suitably carried out in a tray dryer, vacuum oven, Büchi® Rotavapor®, air oven, fluidized bed dryer, spin flash dryer, flash dryer, cone dryer, agitated nutsche filter cum dryer, nauta dryer or the like or any other suitable dryer.

The drying may be carried out at temperature of less than about 150° C., or less than about 120° C., or less than about 100° C., or less than about 70° C., or less than about 60° C., or less than about 50° C., or less than about 40° C., or less than about 20° C., or less than about 0° C., or less than about −20° C. or any other suitable temperature. The drying may be carried out under reduced pressure, that is, less than standard atmospheric pressure or at atmospheric pressure or any other suitable pressure. The drying may take place over a period of about 30 minutes to about 12 hours, or about 2 hours to about 4 hours, or any other suitable time period.

The dried product may be optionally subjected to techniques such as sieving to get rid of lumps before or after drying. The dried product may be optionally milled to get desired particle sizes. Milling or micronization may be performed before drying, or after the completion of drying of the product. Techniques that may be used for particle size reduction include, without limitation, ball, roller and hammer mills, and jet mills.

In an aspect, suvorexant may have a D₉₀ particle size of less than about 200 μm, or less than about 150 μm, or less than about 100 μm, or less than about 90 μm, or less than about 80 μm, or less than about 60 μm, or less than about 50 μm, or less than about 40 μm, or less than about 30 μm, or less than about 20 μm, or less than about 10 μm, or less than about 5 μm, or any other suitable particle sizes.

Particle size distributions of suvorexant particles may be measured using any techniques known in the art. For example, particle size distributions of suvorexant particles may be measured using microscopy or light scattering equipment, such as, for example, a Malvern Master Size 2000 from Malvern Instruments Limited, Malvern, Worcestershire, United Kingdom.

The reactions of the processes described herein can be carried out in air or under an inert atmosphere. Typically, reactions containing reagents or products that are substantially reactive with air can be carried out using air-sensitive synthetic techniques that are well known to the person skilled in art.

For XRD, the relative intensities of the peaks can vary, depending upon the sample preparation technique, the sample mounting procedure and the particular instrument employed. Moreover, instrument variation and other factors can often affect the 2-theta values. Therefore, the peak assignments of diffraction patterns can vary by plus or minus about 0.2°.

In an aspect, the present invention provides a crystalline form of suvorexant, designated as Form A, characterized by an X-ray powder diffraction pattern having peaks expressed in degrees 2θ at about 12.07°, 14.11°, 22.16° and 25.45°±0.2°. It may be further characterized by XRD peaks located at about 18.53° and 19.94°±0.2° 2θ. It may be furthermore characterized by XRD peaks located at about 25.97° and 26.75°±0.2° 2θ.

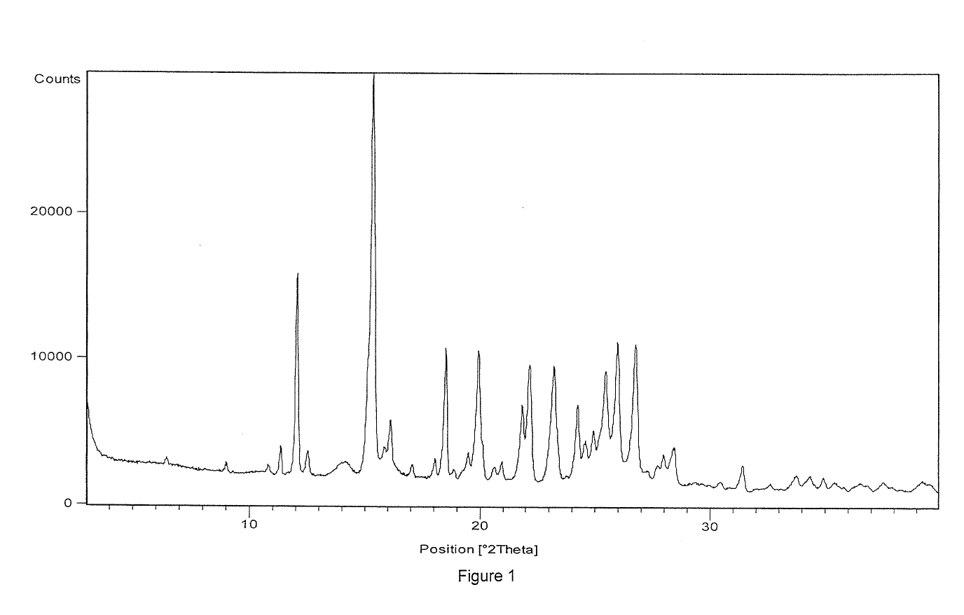

FIG. 1 shows typical X-ray powder diffraction pattern of “Form A”.

In an aspect, the present invention provides a crystalline form of suvorexant, designated as Form A, characterized by an X-ray powder diffraction pattern substantially as illustrated by FIG. 1.

In an aspect, the present invention provides a crystalline form of suvorexant, designated as Form B, characterized by an X-ray powder diffraction pattern having peaks expressed in degrees 2θ located at about 19.93°, 26.73° and 31.38°±0.2°. It may be further characterized by XRD peaks located at about 12.06°, 15.38°, 25.43° and 25.94°±0.2° 2θ. It may be furthermore characterized by XRD peaks located at about 18.53°, 22.16° and 23.21°±0.2° 2θ.

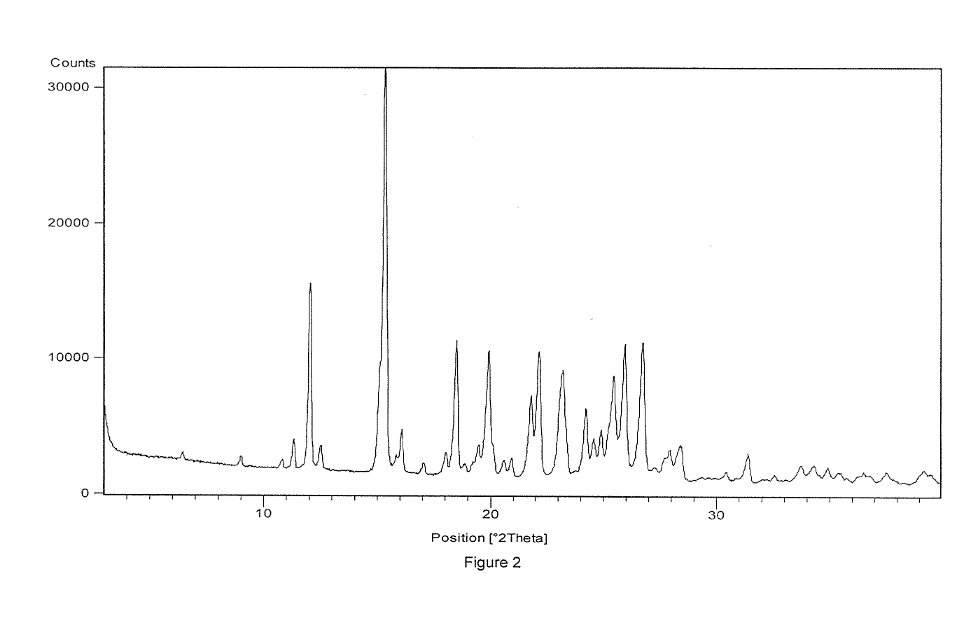

FIG. 2 shows typical X-ray powder diffraction pattern of “Form B”.

In an aspect, the present invention provides a crystalline form of suvorexant, designated as Form B, characterized by an X-ray powder diffraction pattern substantially as illustrated by FIG. 2.

In an aspect, the present invention provides a crystalline form of suvorexant, designated as Form C, characterized by an X-ray powder diffraction pattern having peaks expressed in degrees 2θ located at about 9.70°, 22.20° and 23.91°±0.2°. It may be further characterized by XRD peaks located at about 11.66°, 12.72° and 20.31°±0.2° 2θ. It may be furthermore characterized by XRD peaks located at about 14.16° and 26.47°±0.2° 2θ.

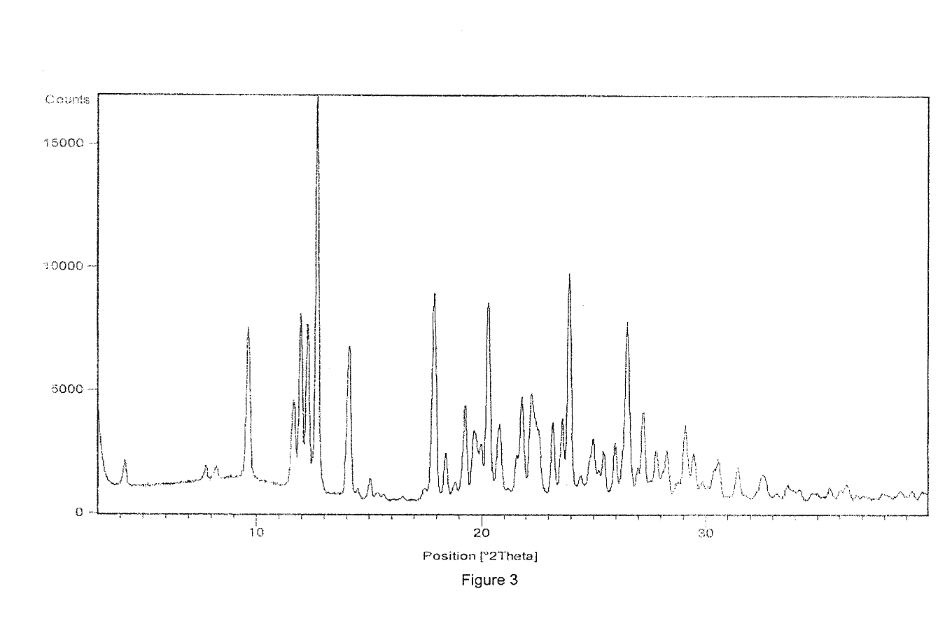

FIG. 3 shows typical X-ray powder diffraction pattern of “Form C”.

In an aspect, the present invention provides a crystalline form of suvorexant, designated as Form C, characterized by an X-ray powder diffraction pattern substantially as illustrated by FIG. 3.

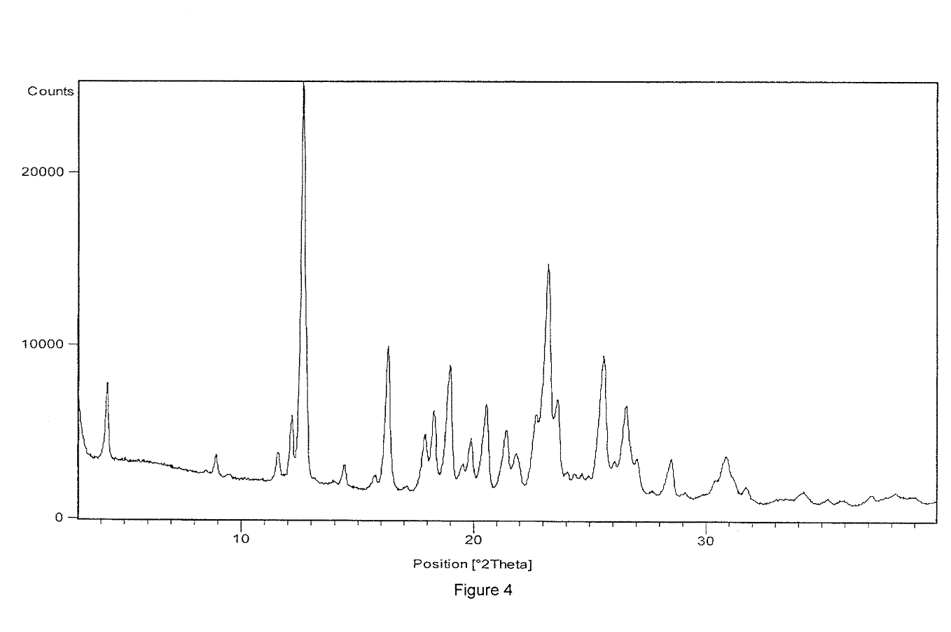

In an aspect, the present invention provides a crystalline form of suvorexant, designated as Form D, characterized by an X-ray powder diffraction pattern having peaks expressed in degrees 2θ located at about 4.26° and 12.66°±0.2°. It may be further characterized by XRD peaks located at about 20.59° and 23.22°±0.2° 2θ. It may be furthermore characterized by XRD peaks located at about 19.01°, 25.62° and 28.51°±0.2° 2θ. FIG. 4 shows typical X-ray powder diffraction pattern of “Form D”.

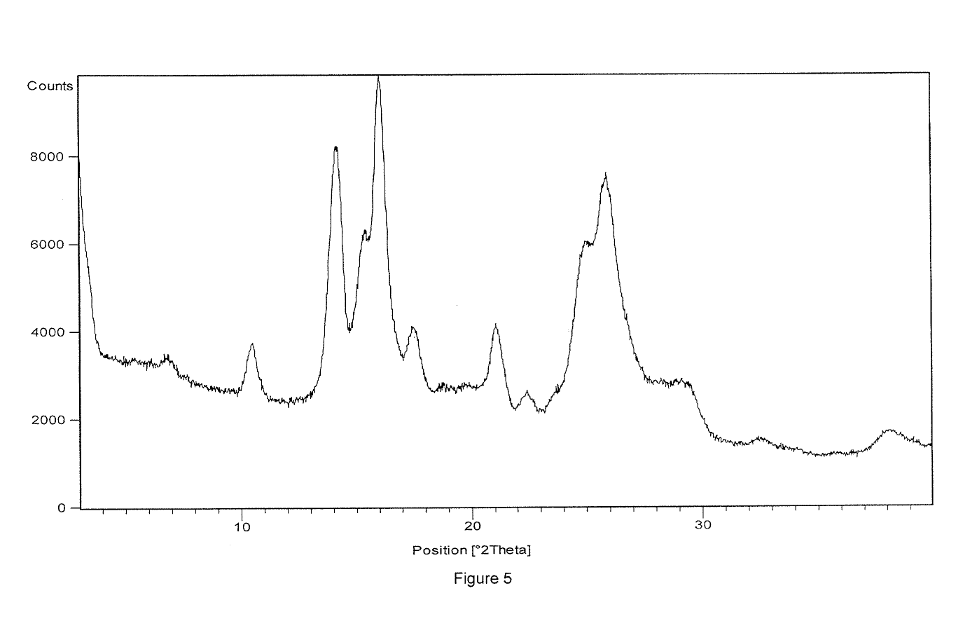

In an aspect, the present invention provides a crystalline form of suvorexant, designated as Form E, characterized by an X-ray powder diffraction pattern having peaks expressed in degrees 2θ located at about 10.53 and 16.05°±0.2°. It may be further characterized by XRD peaks located at about 14.10°, 17.49° and 21.05°±0.2° 2θ. FIG. 5 shows typical X-ray powder diffraction pattern “Form E”.

In an aspect, the present invention provides a crystalline form of suvorexant, designated as Form G, characterized by an X-ray powder diffraction pattern having peaks expressed in degrees 2θ at about 8.14°, 14.97°, 17.92°, 20.25° and 21.74°±0.2° 2θ. It may be further characterized by XRD peaks located at about 4.12°, 9.64°, 14.07°, 22.15°, 23.09° and 23.81°±0.2° 2θ.

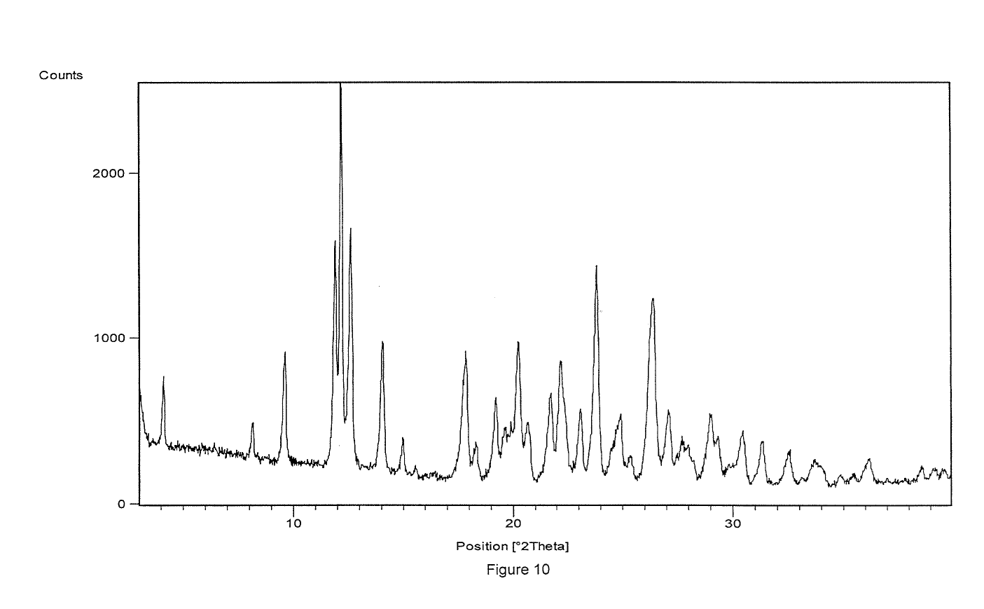

FIG. 10 shows typical X-ray powder diffraction pattern of “Form G”.

In an aspect, the present invention provides a crystalline form of suvorexant, designated as Form G, characterized by an X-ray powder diffraction pattern substantially as illustrated by FIG. 10.

In an aspect, the present invention provides a crystalline form of suvorexant, designated as Form H, characterized by an X-ray powder diffraction pattern having peaks expressed in degrees 2θ at about 7.33°, 9.08°, 11.02°, 14.71°, 22.74° and 29.20°±0.2° 2θ. It may be further characterized by XRD peaks located at about 12.22°, 12.89°, 18.41°, 19.02°, 20.27°, 23.08°, 23.73°, and 26.30°±0.2° 2θ.

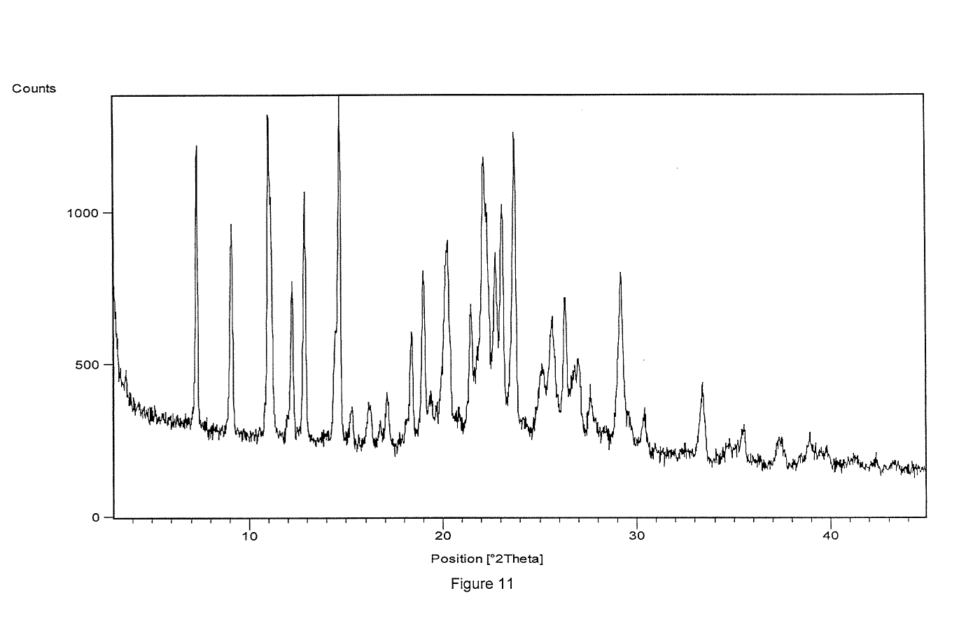

FIG. 11 shows typical X-ray powder diffraction pattern of “Form H”.

In an aspect, the present invention provides a crystalline form of suvorexant, designated as Form H, characterized by an X-ray powder diffraction pattern substantially as illustrated by FIG. 11.

In an aspect, the present invention provides an amorphous form of suvorexant.

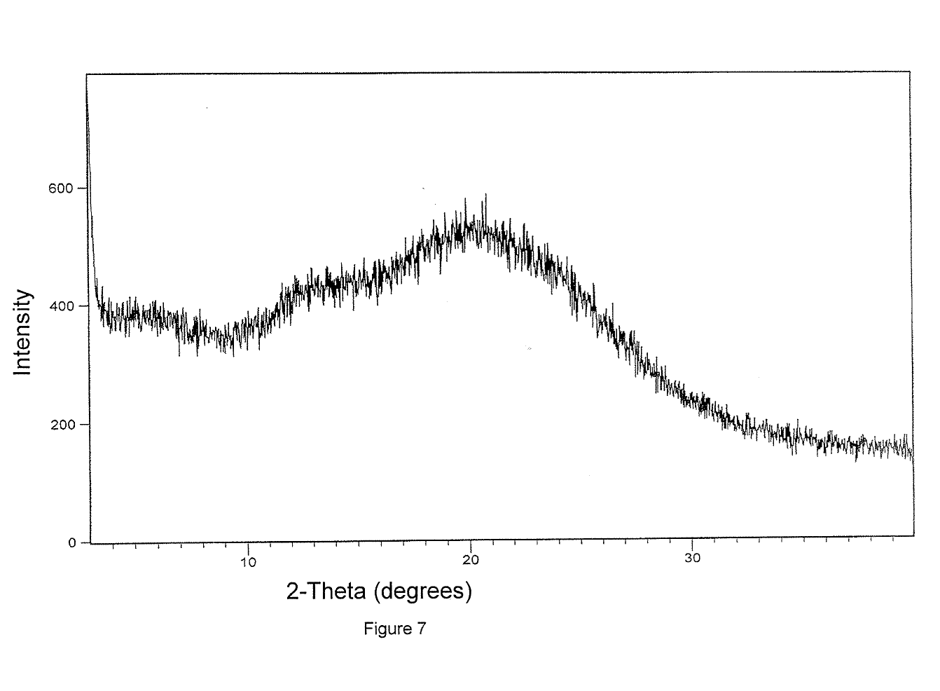

In an aspect, the present invention provides an amorphous form of suvorexant characterized by a powder X-ray diffraction (PXRD) pattern, substantially as illustrated by FIG. 7.

In an aspect, the present invention provides a process for the preparation of amorphous form of suvorexant, comprising:
a) providing a solution of suvorexant in a solvent or mixture of solvents; and
b) isolating amorphous form of suvorexant.

Step a) involves providing a solution of suvorexant in a solvent or mixture of solvents.

Providing a solution in step a) includes:

i) direct use of a reaction mixture containing suvorexant that is obtained in the course of its synthesis; or
ii) dissolving suvorexant in a suitable solvent or mixture of solvents.

Any physical form of suvorexant may be utilized for providing the solution of suvorexant in step a). Suvorexant that may be used as the input for the process of the present invention may be obtained by any process including the processes described in the art. For example, suvorexant may be prepared by the processes described in the United States patent document U.S. Pat. No. 7,951,797 B2 and WIPO publication document WO2012148553A1.

The solvents that may be used for the present invention include, but are not limited to, water, alcohol; ketone; halogenated hydrocarbon; ester; nitrile; polar aprotic solvents; or the like; or mixtures thereof.

The dissolution temperatures may range from about −20° C. to about reflux temperature of the solvent, depending on the solvent used for dissolution, as long as a clear solution of suvorexant is obtained without affecting its quality. The solution may optionally be treated with carbon, flux-calcined diatomaceous earth (Hyflow), or any other suitable material to remove color and/or to clarify the solution.

Optionally, the solution obtained above may be filtered to remove any insoluble particles. The insoluble particles may be removed suitably by filtration, centrifugation, decantation, or any other suitable techniques. The solution may be filtered by passing through paper, glass fiber, or other membrane material, or a bed of a clarifying agent such as Celite® or Hyflow. Depending upon the equipment used and the concentration and temperature of the solution, the filtration apparatus may need to be preheated to avoid premature crystallization.

Step b) involves isolation of amorphous form of suvorexant from the solution of step a).

The isolation may be effected by removing the solvent. Suitable techniques which may be used for the removal of the solvent include evaporation techniques such as a Büchi® Rotavapor®, spray drying, agitated thin film drying, freeze drying (lyophilization) or the like or any other suitable technique.

The solvent may be removed, optionally under reduced pressures, at temperatures less than about 150° C., less than about 100° C., less than about 60° C., less than about 40° C., less than about 20° C., less than about 0° C., less than about −20° C., less than about −40° C., less than about −60° C., less than about −80° C., or any other suitable temperatures. The solvent may be removed optionally at atmospheric pressure at temperatures such as described above in this paragraph.

Freeze drying (lyophilization) may be carried out by freezing a solution of suvorexant at low temperatures and reducing the pressure required to remove the solvent from the frozen solution of suvorexant. Temperatures that may be required to freeze the solution, depending on the solvent chosen to make the solution of suvorexant, may range from about −80° C. to about 0° C., or up to about 20° C. Temperatures that may be required to remove the solvent from the frozen solution may be less than about 20° C., less than about 0° C., less than about −20° C., less than about −40° C., less than about −60° C., less than about −80° C., or any other suitable temperatures.

In another embodiment, isolation may also be effected by combining the solution of step a) with a suitable anti-solvent or combining an anti-solvent to the solution obtained in step a) rapidly. Anti-solvent as used herein refers to a solvent in which suvorexant is poorly soluble. Suitable anti-solvents that may be used include, but are not limited to: water, aliphatic or alicyclic hydrocarbon; substituted hydrocarbons such as nitromethane; aromatic hydrocarbon; ether; or mixtures thereof.

The compound obtained from step b) may be collected using techniques such as scraping, or shaking the container, or other techniques specific to the equipment used.

In case of isolation using anti-solvent described above, step b) may be followed by isolation techniques including filtration, decantation, centrifugation, gravity filtration, suction filtration or any other techniques for the recovery of the solids. For filtration, equipments such as nutsche filter, centrifuge, agitated nutsche filter, leaf filter or any other suitable equipment for filtration may be used.

The product thus isolated may be optionally further dried to afford an amorphous form of suvorexant.

In an aspect, the present invention provides a solid dispersion of suvorexant together with one or more pharmaceutically acceptable carriers, excipients, or diluents.

In an aspect, the present invention provides an amorphous solid dispersion of suvorexant together with one or more pharmaceutically acceptable carriers or excipients.

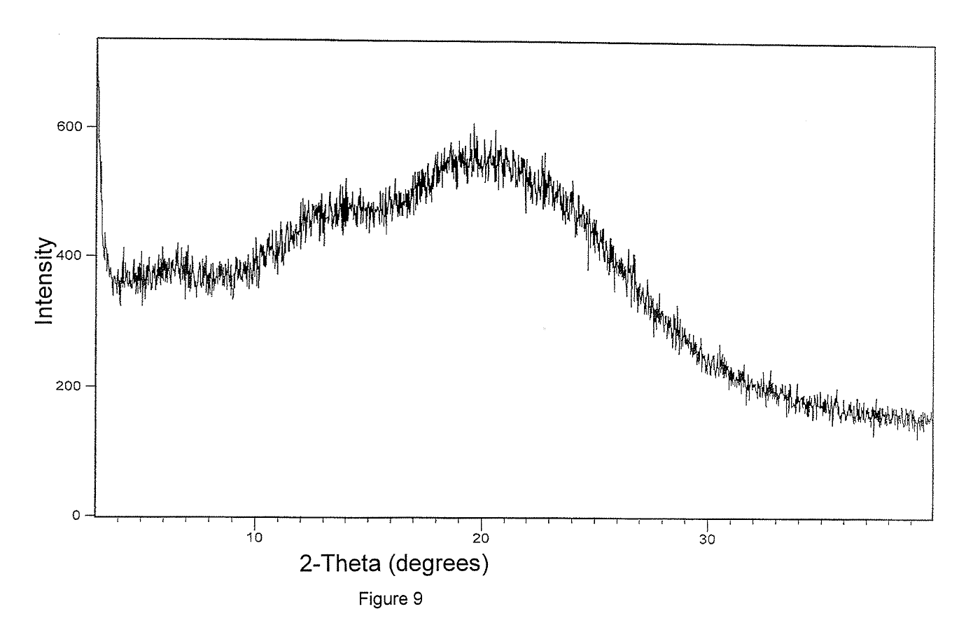

In an aspect, the present invention provides an amorphous solid dispersion of suvorexant together with one or more pharmaceutically acceptable carriers or excipients characterized by a powder X-ray diffraction (PXRD) pattern, substantially as illustrated by FIG. 9.

In an aspect, the present invention provides a process for preparing a solid dispersion of suvorexant together with one or more pharmaceutically acceptable carriers or excipients, comprising:

a) providing a solution or suspension of suvorexant in combination with one or more pharmaceutically acceptable carriers or excipients in a solvent or mixture of solvents; and
b) isolating a solid dispersion of suvorexant together with one or more pharmaceutically acceptable carriers or excipients.

In an aspect, the present invention provides a process for preparing amorphous solid dispersion of suvorexant together with one or more pharmaceutically acceptable carrier or excipients comprising:

a) providing a solution or suspension of suvorexant in combination with one or more pharmaceutically acceptable carriers or excipients in a solvent or mixture of solvents; and
b) isolating amorphous solid dispersion of suvorexant together with one or more pharmaceutically acceptable carriers or excipients.

Step a) involves providing a solution or suspension of suvorexant optionally in combination with one or more pharmaceutically acceptable carriers or excipients in a solvent or mixture of solvents.

In aspects, step a) may involve forming a solution of suvorexant together with one or more pharmaceutically acceptable carriers or excipients. In aspects, carriers or excipients enhance stability of the amorphous solid upon removal of the solvent.

Providing the solution in step a) includes:

i) direct use of a reaction mixture containing suvorexant that is obtained in the course of its manufacture, if desired, after addition of one or more pharmaceutically acceptable carriers or excipients; or
ii) dissolution of suvorexant in a suitable solvent, either alone or in combination with one or more pharmaceutically acceptable carriers or excipients.

Any physical form of suvorexant may be utilized for providing a solution in step a).

Pharmaceutically acceptable carriers that may be used for the preparation of solid dispersions of suvorexant of the present invention include, but are not limited to: water soluble sugar derivatives including any pharmaceutically acceptable water soluble sugar excipients, preferably having low hygroscopicity, which include, but are not limited to, mannitol, lactose, fructose, sorbitol, xylitol, maltodextrin, dextrates, dextrins, lactitol, or the like; pharmaceutical hydrophilic carriers such as polyvinylpyrrolidones (homopolymers or copolymers of N-vinyl pyrrolidone), gums, cellulose derivatives (including hydroxypropyl methylcelluloses, hydroxypropyl celluloses, microcrystalline celluloses and others), polymers of carboxymethyl celluloses, cyclodextrins, gelatins, hypromellose phthalates, sugars, polyhydric alcohols, polyethylene glycols, polyethylene oxides, polyoxyethylene derivatives, polyvinyl alcohols, propylene glycol derivatives, or the like; or organic amines such as alkyl amines (primary, secondary, and tertiary), aromatic amines, alicyclic amines, cyclic amines, aralkyl amines, hydroxylamine or its derivatives, hydrazine or its derivatives, and guanidine or its derivatives. The use of mixtures of more than one of the pharmaceutical excipients to provide desired release profiles or for the enhancement of stability is within the scope of this invention. Also, all viscosity grades, molecular weights, commercially available products, their copolymers, and mixtures are all within the scope of this invention without limitation.

Any suitable quantity of excipient or carrier may be used for the preparation of the said solid dispersion of suvorexant in combination with one or more pharmaceutically acceptable carriers. For example, about 0.1, about 0.5, about 1.0, about 2.0, about 3.0, about 4.0, about 5.0, about 6.0, about 7.0, about 8.0, about 9.0 or about 10.0 portions by weight of excipient or carrier can be used per one portion of suvorexant.

When the solution or suspension of suvorexant is prepared together with a pharmaceutically acceptable carrier, the order of charging different materials to the solution is not critical for obtaining the desired solid dispersion. A specific order may be preferred with respect to the equipment being used and will be easily determined by a person skilled in the art. Suvorexant or pharmaceutically acceptable carrier may be completely soluble in the solvent or they may form a suspension. In embodiments, suvorexant and the pharmaceutically acceptable carrier may be separately dissolved either in the same solvent or in different solvents, and then combined to form a mixture.

Suitable solvents that may be used in step a) include, but are not limited to, water, alcohol; ketone; halogenated hydrocarbon; ester; nitrile; polar aprotic; or mixtures thereof.

The dissolution temperatures may range from about −20° C. to about the reflux temperature of the solvent, depending on the solvent used for dissolution, as long as a clear solution of suvorexant is obtained without affecting its quality. The solution may optionally be treated with carbon, flux-calcined diatomaceous earth (Hyflow), or any other suitable material to remove color and/or to clarify the solution.

Optionally, the solution obtained above may be filtered to remove any insoluble particles. The insoluble particles may be removed suitably by filtration, centrifugation, decantation, or any other suitable techniques. The solution may be filtered by passing through paper, glass fiber, or other membrane material, or a bed of a clarifying agent such as Celite® or Hyflow. Depending upon the equipment used and the concentration and temperature of the solution, the filtration apparatus may need to be preheated to avoid premature crystallization.

Step b) involves isolation of amorphous solid dispersion of suvorexant together with one or more pharmaceutically acceptable carriers from the solution of step a).

The isolation may be effected by removing the solvent. Suitable techniques which may be used for the removal of the solvent include evaporation techniques such as a Büchi® Rotavapor®, spray drying, agitated thin film drying, freeze drying (lyophilization) and the like or any other suitable technique. The solvent may be removed, optionally under reduced pressures, at temperatures less than about 150° C., less than about 100° C., less than about 60° C., less than about 40° C., less than about 20° C., less than about 0° C., less than about −20° C., less than about −40° C., less than about −60° C., less than about −80° C., or any other suitable temperatures. The solvent may be removed optionally at atmospheric pressure at temperatures such as described above in this paragraph.

Freeze drying (lyophilization) may be carried out by freezing a solution of suvorexant at low temperatures and reducing the pressure required to remove the solvent from the frozen solution of suvorexant. Temperatures that may be required to freeze the solution, depending on the solvent chosen to make the solution of suvorexant, may range from about −80° C. to about 0° C., or up to about 20° C. Temperatures that may be required to remove the solvent from the frozen solution may be less than about 20° C., less than about 0° C., less than about −20° C., less than about −40° C., less than about −60° C., less than about −80° C., or any other suitable temperatures.

In another aspect, isolation may also be effected by combining the solution of step a) comprising one or more pharmaceutically acceptable carriers with a suitable anti-solvent. Combining the solution obtained in step a) to the anti-solvent, or combining an anti-solvent to the solution obtained in step a), both within the scope of the present invention. Suitable anti-solvents that may be used include, but are not limited to: water, aliphatic or alicyclic hydrocarbon solvents; substituted hydrocarbon solvents such as nitromethane; aromatic hydrocarbon solvents; ether solvents; or mixtures thereof.

The compound obtained from step b) may be collected using techniques such as scraping, or shaking the container, or other techniques specific to the equipment used. In case of isolation using anti-solvent described above, step b) may be followed by isolation techniques including filtration, decantation, centrifugation, gravity filtration, suction filtration or any other techniques for the recovery of the solids. For filtration, equipments such as nutsche filter, centrifuge, agitated nutsche filter, leaf filter or any other suitable equipment for filtration may be used.

The product thus isolated may be optionally further dried to afford an amorphous form of suvorexant.

Examples of an amorphous solid dispersions of suvorexant together with a pharmaceutically acceptable carrier obtained using the above process are characterized by their powder X-ray diffraction (“PXRD”) patterns substantially as illustrated by FIG. 9 respectively.

The solid dispersions differ from physical mixtures of amorphous suvorexant and one or more pharmaceutically acceptable carriers, so that individual particles of the components cannot be distinguished using techniques such as optical microscopy. In instances, the solid dispersions contain the components on a molecular level, such as in the nature of solid solutions.

In an aspect, the present invention provides a pharmaceutical composition comprising amorphous form of suvorexant along with one or more pharmaceutically acceptable carriers, excipients, or diluents.

In an aspect, the present invention provides pharmaceutical formulations comprising a solid dispersion of suvorexant together with one or more pharmaceutically acceptable carriers, excipients, or diluents.

In an aspect, the present invention provides pharmaceutical formulations comprising an amorphous solid dispersion of suvorexant together with one or more pharmaceutically acceptable carriers, excipients, or diluents.

A solid dispersion of suvorexant together with one or more pharmaceutically acceptable excipients of the present invention may be further formulated as: solid oral dosage forms such as, but not limited to: powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the forms of immediate release, delayed release or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems. The compositions may be prepared using techniques such as direct blending, dry granulation, wet granulation, and extrusion and spheronization. Compositions may be presented as uncoated, film coated, sugar coated, powder coated, enteric coated, and modified release coated. Compositions of the present invention may further comprise one or more pharmaceutically acceptable excipients.

Polymorphic forms of present invention may be prepared by utilizing following general methods:

Any polymorphic form may be taken as starting material to prepare desired polymorph.

### Method A

The process comprises mixing suvorexant with a suitable solvent or mixture of solvents and stirring at a suitable temperature until suvorexant is dissolved completely; precipitating suvorexant by cooling and isolating the obtained precipitates. Optionally, isolated precipitates are further dried. If necessary, the solvent may be heated to effect dissolution of suvorexant. Suitable temperature for dissolution is preferably between room temperature and reflux temperature of the solvent used for the dissolution. The precipitation comprises cooling and optionally aging the mixture. The cooling may be done to a temperature of less than about 50° C., or less than about 40° C., or less than about 30° C., or less than about 20° C., or less than about 10° C., or less than about 5° C., or less than about 0° C., or less than about −5° C., or any other suitable temperature. The aging may be typically done for about 24 hours or about 18 hours or about 12 hours or about 10 hours or about 5 hours or about 2 hours or about 1 hour or any other suitable time period. The crystalline form may be recovered, e.g., by filtration, drying, decantation, centrifugation, gravity filtration, suction filtration or any other suitable techniques for the recovery of solids.

### Method B

Suvorexant is dissolved in a suitable solvent or mixture of solvents to obtain a solution, and the solution is then added to an anti-solvent. Alternatively, anti-solvent may be added to the solution of suvorexant. Precipitates are isolated and optionally further dried. When the solution containing suvorexant dissolved in solvent is mixed with anti-solvent, suvorexant precipitates, it is not necessary to wait for a certain amount of time after mixing; however, crystallization may require aging for additional time. Aging is typically done at a temperature of less than about 50° C., or less than about 40° C., or less than about 30° C., or less than about 20° C., or less than about 10° C., or less than about 5° C., or less than about 0° C., or less than about −5° C., or any other suitable temperature. The aging may be typically done for about 24 hours, or about 18 hours, or about 12 hours, or about 10 hours, or about 5 hours, or about 2 hours, or about 1 hour, or any other suitable time period. The crystalline form may be recovered, e.g., by filtration, drying, decantation, centrifugation, gravity filtration, suction filtration or any other techniques for the recovery of the solids.

The anti-solvent as used herein refers to a solvent in which suvorexant at the temperature at which the process is performed is less soluble than in the solvent, so that suvorexant crystallizes when it is mixed with the anti-solvent. The solvent used in the dissolution of suvorexant is miscible with anti-solvent. A solvent is miscible with the anti-solvent, if, the temperature at which process is carried out they form a homogenous solvent mixture.

### Method C

The process comprises mixing suvorexant with a suitable solvent or mixture of solvents to get heterogeneous mixture and slurrying the mixture at room temperature or less than room temperature or any other suitable temperature for a time period of about 24 hours, or about 18 hours, or about 12 hours, or about 10 hours, or about 5 hours, or about 2 hours, or about 1 hour, or any other suitable time period. The crystalline form may be recovered, e.g., by filtration, drying, decantation, centrifugation, gravity filtration, suction filtration or any other techniques for the recovery of the solids.

### Method D

Suvorexant is dissolved in a suitable solvent or mixture of solvents and vessel containing solution is kept aside for slow solvent evaporation. If necessary, the solvent may be heated to effect dissolution of suvorexant.

### Method E

Suvorexant is dissolved in a suitable solvent or mixture of solvents and the contents may be heated, if necessary, to effect dissolution of suvorexant. The resulting solution is subjected to fast or flash evaporation. The evaporation may be carried out at atmospheric pressure or under reduced pressure or under pressure.

Desired polymorphic form may also be prepared by the combination of two or more methods.

Purity of suvorexant may be measured by following HPLC method as depicted here under:

Apparatus: A liquid chromatography equipped with PDA detector
Column: Reverse phase RP-18 column with hybrid particle

### Wavelength: 215 nm

HPLC method is with linear gradient elution having mobile phase A as 0.05M aqueous ammonium acetate and mobile phase B as acetonitrile.

According to another aspect of the present invention there is provided a pharmaceutical composition comprising one or more of Form A, Form B, Form C, Form D, Form E, Form G, Form H and amorphous form of suvorexant and pharmaceutically acceptable carrier.

The pharmaceutical composition one or more of Form A, Form B, Form C, Form D, Form E, Form G, Form H and amorphous form of suvorexant together with one or more pharmaceutically acceptable excipients of the present invention may be further formulated as: solid oral dosage forms such as, but not limited to: powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the forms of immediate release, delayed release or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems. The compositions may be prepared using techniques such as direct blending, dry granulation, wet granulation, and extrusion and spheronization. Compositions may be presented as uncoated, film coated, sugar coated, powder coated, enteric coated, and modified release coated. Compositions of the present invention may further comprise one or more pharmaceutically acceptable excipients.

Pharmaceutically acceptable excipients that are useful in the present invention include, but are not limited to: diluents such as starches, pregelatinized starches, lactose, powdered celluloses, microcrystalline celluloses, dicalcium phosphate, tricalcium phosphate, mannitol, sorbitol, sugar or the like; binders such as acacia, guar gum, tragacanth, gelatin, polyvinylpyrrolidones, hydroxypropyl celluloses, hydroxypropyl methylcelluloses, pregelatinized starches or the like; disintegrants such as starches, sodium starch glycolate, pregelatinized starches, crospovidones, croscarmellose sodium, colloidal silicon dioxide or the like; lubricants such as stearic acid, magnesium stearate, zinc stearate or the like; glidants such as colloidal silicon dioxide or the like; solubility or wetting enhancers such as anionic or cationic or neutral surfactants; complex forming agents such as various grades of cyclodextrins and resins; release rate controlling agents such as hydroxypropyl celluloses, hydroxymethyl celluloses, hydroxypropyl methylcelluloses, ethylcelluloses, methylcelluloses, various grades of methyl methacrylates, waxes or the like. Other pharmaceutically acceptable excipients that are of use include but are not limited to film formers, plasticizers, colorants, flavoring agents, sweeteners, viscosity enhancers, preservatives, antioxidants, or the like.

### DEFINITIONS

The following definitions are used in connection with the present invention unless the context indicates otherwise. The term “amorphous” refers to a solid lacking any long-range translational orientation symmetry that characterizes crystalline structures although, it may have short range molecular order similar to a crystalline solid. The term “anti-solvent” refers to a liquid that, when combined with a solution of suvorexant, reduces solubility of the suvorexant in the solution, causing crystallization or precipitation in some instances spontaneously, and in other instances with additional steps, such as seeding, cooling, scratching and/or concentrating. Celite® is flux-calcined diatomaceous earth. Celite® is a registered trademark of World Minerals Inc. Hyflow is flux-calcined diatomaceous earth treated with sodium carbonate. Hyflo Super Cel™ is a registered trademark of the Manville Corp. Polymorphs are different solids sharing the same molecular formula, yet having distinct physical properties when compared to other polymorphs of the same formula.

Suitable solvent as used herein include, but are not limited to, alcohol, aliphatic hydrocarbon, alicyclic hydrocarbon, aromatic hydrocarbon, halogenated hydrocarbon, ester, ether, nitrile, polar aprotic, ketone, or mixtures thereof.

An “alcohol solvent” is an organic solvent containing a carbon bound to a hydroxyl group. “Alcoholic solvents” include, but are not limited to, methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, glycerol, C₁₋₆ alcohols, or mixtures thereof.

An “aliphatic or alicyclic hydrocarbon solvent” refers to a liquid, non-aromatic, hydrocarbon, which may be linear, branched, or cyclic. It is capable of dissolving a solute to form a uniformly dispersed solution. Examples of a hydrocarbon solvents include, but are not limited to, n-pentane, isopentane, neopentane, n-hexane, isohexane, 3-methylpentane, 2,3-dimethylbutane, neohexane, n-heptane, isoheptane, 3-methylhexane, neoheptane, 2,3-dimethylpentane, 2,4-dimethylpentane, 3,3-dimethylpentane, 3-ethylpentane, 2,2,3-trimethylbutane, n-octane, isooctane, 3-methylheptane, neooctane, cyclohexane, methylcyclohexane, cycloheptane, C₅-C₈ aliphatic hydrocarbons, petroleum ethers, or mixtures thereof.

“Aromatic hydrocarbon solvent” refers to a liquid, unsaturated, cyclic, hydrocarbon containing one or more rings which has at least one 6-carbon ring containing three double bonds. It is capable of dissolving a solute to form a uniformly dispersed solution. Examples of aromatic hydrocarbon solvents include, but are not limited to, benzene toluene, ethylbenzene, m-xylene, o-xylene, p-xylene, indane, naphthalene, tetralin, trimethylbenzene, chlorobenzene, fluorobenzene, trifluorotoluene, anisole, C6-C10 aromatic hydrocarbons, or mixtures thereof.

An “ester solvent” is an organic solvent containing a carboxyl group —(C═O)—O— bonded to two other carbon atoms. “Ester solvents” include, but are not limited to, ethyl acetate, n-propyl acetate, n-butyl acetate, isobutyl acetate, t-butyl acetate, ethyl formate, methyl acetate, methyl propanoate, ethyl propanoate, methyl butanoate, ethyl butanoate, C₃₋₆ esters, or mixtures thereof.

A “halogenated hydrocarbon solvent” is an organic solvent containing a carbon bound to a halogen. “Halogenated hydrocarbon solvents” include, but are not limited to, dichloromethane, 1,2-dichloroethane, trichloroethylene, perchloroethylene, 1,1,1-trichloroethane, 1,1,2-trichloroethane, chloroform, carbon tetrachloride, or mixtures thereof.

A “ketone solvent” is an organic solvent containing a carbonyl group —(C═O)— bonded to two other carbon atoms. “Ketone solvents” include, but are not limited to, acetone, ethyl methyl ketone, diethyl ketone, methyl isobutyl ketone, C₃₋₆ ketones, 4-methylpentane-2-one or mixtures thereof.

A “nitrile solvent” is an organic solvent containing a cyano —(C≡N) bonded to another carbon atom. “Nitrile solvents” include, but are not limited to, acetonitrile, propionitrile, C₂₋₆ nitriles, or mixtures thereof.

A “polar aprotic solvent” has a dielectric constant greater than 15 and is at least one selected from the group consisting of amide-based organic solvents, such as N,N-dimethylformamide (DMF), N,N-dimethylacetamide (DMA), N-methylpyrrolidone (NMP), formamide, acetamide, propanamide, hexamethyl phosphoramide (HMPA), and hexamethyl phosphorus triamide (HMPT); nitro-based organic solvents, such as nitromethane, nitroethane, nitropropane, and nitrobenzene; pyridine-based organic solvents, such as pyridine and picoline; sulfone-based solvents, such as dimethylsulfone, diethylsulfone, diisopropylsulfone, 2-methylsulfolane, 3-methylsulfolane, 2,4-dimethylsulfolane, 3,4-dimethy sulfolane, 3-sulfolene, and sulfolane; and sulfoxide-based solvents such as dimethylsulfoxide (DMSO).

An “ether solvent” is an organic solvent containing an oxygen atom —O— bonded to two other carbon atoms. “Ether solvents” include, but are not limited to, diethyl ether, diisopropyl ether, methyl t-butyl ether, glyme, diglyme, tetrahydrofuran, 2-methyltetrahydrofuran, 1,4-dioxane, dibutyl ether, dimethylfuran, 2-methoxyethanol, 2-ethoxyethanol, anisole, C₂₋₆ ethers, or the like.

Certain specific aspects and embodiments of the present invention will be explained in more detail with reference to the following examples, which are provided for purposes of illustration only and should not be construed as limiting the scope of the present invention in any manner.

As can be observed from the PXRD pattern of Forms A and B, the difference between these two forms is only in the additional characteristic peak located at about 14.11 degrees 2-theta in Form A when compared to the characteristic peaks of Form B. Further, Form A is getting converted in to Form B up on slurrying in few solvents (Examples 26 to 38). Therefore, without binding by any theory, it is believed that Form A could be a mixture of Form B and another form.

### EXAMPLES

All PXRD data reported herein are obtained using a Bruker AXS D8 Advance Powder X-ray Diffractometer or a PANalytical X-ray Diffractometer, using copper Kα radiation wavelength 1.5418 Å.

### Example 1

Suvorexant (500 mg) was taken in ethyl acetate (14 ml) and heated at 75° C. to get clear solution. The resulting solution was slowly cooled to 0-5° C. and then stirred for 16 hour. The resulting crystals were filtered and then dried in air tray drier for 30 minute at 60° C. to obtain Form A.

### Example 2

Suvorexant (500 mg) was taken in acetone (10 ml) and heated at 60° C. to get clear solution. The resulting solution was cooled to 28° C. and then stirred for 18 hour. The resulting crystals were filtered and then dried under vacuum to obtain Form A.

### Example 3

Suvorexant (500 mg) was taken in 2-butanol (15 ml) and heated to get clear solution. The resulting solution was cooled to 27° C. and stirred for 2 hour then further cooled to 0° C. over 2 hour. The precipitated product was filtered and then dried under vacuum at 50° C. to obtain Form A.

### Example 4

Suvorexant Form A (500 mg) and tert-butyl alcohol (10 ml) were introduced in 100 ml round bottom flask. The heterogeneous mixture so obtained was slurried with stirring at about 27° C. for about 17 hour and the solid substance was then filtered under vacuum. The product was dried in air tray drier at 50° C. for 2 hour to obtain Form A.

### Example 5-7

The process of example 4 was repeated wherein tert-butyl alcohol was replaced by following solvents to obtain Form A:

| Example | Solvent | Quantity |
|---|---|---|
| Example 5 | Ethylene Glycol | 20 ml |
| Example 6 | Heptane | 20 ml |
| Example 7 | Water | 20 ml |

### Example 8

Suvorexant (50 mg) was taken in acetone (30 ml) and heated at 60° C. to get clear solution. Vessel containing the resulting solution was kept aside for slow solvent evaporation at 27° C. to obtain Form A.

### Example 9-13

The process of example 8 was repeated wherein acetone was replaced by following solvents to obtain Form A:

| Example | Solvent | Quantity |
|---|---|---|
| Example 9 | Ethyl acetate | 18 ml |
| Example 10 | Ethanol | 20 ml |
| Example 11 | Acetonitrile | 20 ml |
| Example 12 | Methyl isobutyl | 18 ml |
| | ketone (MIBK) | |
| Example 13 | Butyl acetate | 20 ml |

### Example 14

Suvorexant (500 mg) was dissolved under stirring at 65° C. in DMF (6 ml). The resulting solution was cooled to room temperature and stirred for 1 hour. The solution was further cooled to 0-5° C. and stirred for 1 hour. The temperature of solution was raised to room temperature and stirred for 14 hours. Water (20 ml) was charged in to the solution. After the addition of water to the solution was complete, resulting mixture was stirred for 30 minute. The mixture was filtered under vacuum and dried at 50° C. to obtain Form A.

### Example 15

Suvorexant (500 mg) was dissolved under stirring at 60° C. in THF (5 ml). The resulting solution was cooled to room temperature and stirred for 18 hours. Water (20 ml) was charged in to the solution. After the addition of water to the solution was complete, resulting mixture was stirred for about 30 minute. The mixture was filtered under vacuum and dried at 50° C. in air tray drier to obtain Form A.

### Example 16

Suvorexant (500 mg) was dissolved under stirring at 56° C. in acetone (40 ml). The resulting solution was cooled to room temperature and added slowly to water (80 ml). After the addition of the solution of suvorexant to water was complete, resulting mixture was stirred for 2 hours. The mixture was filtered under vacuum and dried at 60° C. in air tray drier to obtain Form A.

### Example 17

Suvorexant (500 mg) was dissolved under stirring at 55° C. in THF (15 ml). The resulting solution was cooled to room temperature and added slowly to water (45 ml). After the addition of the solution of suvorexant to water was complete, resulting mixture was stirred for 2 hours. The mixture was filtered under vacuum and dried at 60° C. in air tray drier to obtain Form A.

### Example 18

Suvorexant (500 mg) was dissolved under stirring at 60° C. in methanol (25 ml). The resulting solution was cooled to room temperature and added slowly to water (50 ml). After the addition of the solution of suvorexant to water was complete, resulting mixture was stirred for 18 hours. The mixture was filtered under vacuum and dried at 60° C. in air tray drier to obtain Form A.

### Example 19

Suvorexant (500 mg) was dissolved under stirring at 60° C. in acetonitrile (25 ml). The resulting solution was cooled to room temperature and added slowly to water (50 ml). After the addition of the solution of suvorexant to water was complete, resulting mixture was stirred for 18 hours. The mixture was filtered under vacuum and dried at 60° C. in air tray drier to obtain Form A.

### Example 20

Suvorexant (2 gm) was dissolved under stirring at 70° C. in n-propanol (160 ml). The resulting solution was cooled to room temperature and added slowly to water (320 ml). After the addition of the solution of suvorexant to water was complete, resulting mixture was cooled to 5° C. and stirred for 2 hours. The mixture was filtered under vacuum and dried at 40° C. for 3 hour in air tray drier to obtain Form A.

### Example 21

Suvorexant (500 mg) was dissolved under stirring at 60° C. in methanol (25 ml). The resulting solution was cooled to room temperature and added slowly to n-heptane (50 ml). After the addition of the solution of suvorexant to heptane was complete, resulting mixture was stirred for about 23 hour. The mixture was filtered under vacuum and dried at 50° C. in air tray drier to obtain Form A.

### Example 22

Suvorexant (500 mg) was dissolved under stirring at 80° C. in n-propanol (40 ml). The resulting solution was cooled to room temperature and added slowly to n-heptane (120 ml). After the addition of the solution of suvorexant to heptane was complete, resulting mixture was stirred for about 30 minute at 0° C. The mixture was filtered under vacuum and dried at 70° C. in air tray drier to obtain Form A.

### Example 23

Suvorexant (500 mg, HPLC Purity 99.03%) and methanol (30 ml) were introduced in round bottom flask and heated to 60° C. to get clear solution. Methanol was distilled out from the resulting solution under vacuum to obtain amorphous suvorexant. Ethyl acetate (2.5 ml) was charged to the solid, followed by addition of hexane (5 ml) and stirred for about 24 hour. The product was filtered and dried at 55° C. in air tray drier for 1 hour to obtain Form A (HPLC Purity 99.78%).

### Example 24

Suvorexant (500 mg, HPLC Purity 99.03%) and acetonitrile (30 ml) were introduced in round bottom flask and heated to 60° C. to get clear solution. Acetonitrile was distilled out from the resulting solution under vacuum to obtain amorphous suvorexant. Ethyl acetate (2.5 ml) was charged to the solid, followed by addition of hexane (5 ml) and stirred for about 24 hour. The product was filtered and dried in air tray drier at 55° C. for 1 hour to obtain Form A (HPLC Purity 99.42%).

### Example 25

Suvorexant (500 mg) and acetone (30 ml) were introduced in round bottom flask and heated to 60° C. to get clear solution. Acetone was distilled out from the resulting solution under vacuum to obtain amorphous suvorexant. Ethyl acetate (2.5 ml) was charged to the solid, followed by addition of hexane (5 ml) and stirred for about 24 hour. The product was filtered and dried at 55° C. in air tray drier for 1 hour to obtain Form A.

### Example 26

Suvorexant Form-A (500 mg) and methanol (20 ml) were introduced in 100 ml round bottom flask. The heterogeneous mixture so obtained was stirred at 27° C. for 17 hours and the solid substance was then filtered under vacuum. The product was dried at 60° C. in air tray drier to obtain Form B.

### Example 27-38

The process of example 26 was repeated wherein methanol was replaced by following solvents to obtain Form B:

| | | | | | | Drying | | |
|---|---|---|---|---|---|---|---|---|
| Example | Solvent | Quantity | | Stirring time | | Temperature | Time | |
| Example 27 | Acetone | 5 | ml | 17 | hours | 60° C. | 2 | hours |
| Example 28 | Dimethyl carbonate | 5 | ml | 17 | hours | 60° C. | 2 | hours |
| | | | | 10 | minutes | | 20 | minutes |
| Example 29 | Isoamyl alcohol | 10 | ml | 17 | hours | 60° C. | 1 | hour |
| | | | | 35 | minutes | | 40 | minutes |
| Example 30 | Isobutanol | 10 | ml | 17 | hours | 60° C. | 2 | hours |
| | | | | 50 | minutes | | | |
| Example 31 | Isopropyl alcohol | 20 | ml | 17 | hours | 50° C. | 2 | hours |
| | | | | | | | 10 | minutes |
| Example 32 | Propanol | 10 | ml | 17 | hours | 28° C. | 2 | hours |
| | | | | | | | 10 | minutes |
| Example 33 | Cyclohexanol | 25 | ml | 17 | hours | 50° C. | 2 | hours |
| | | | | 10 | minutes | | 10 | minutes |
| Example 34 | 2-butanol | 10 | ml | 17 | hours | 20° C. | 2 | hours |
| | | | | 15 | minutes | | | |
| Example 35 | Acetonitrile | 30 | ml | 24 | hours | 50° C. | 2 | hours |
| | | | | | | | 10 | minutes |
| Example 36 | Ethyl acetate | 30 | ml | 24 | hours | 50° C. | 2 | hours |
| | | | | 10 | minutes | | 10 | minutes |
| Example 37 | Dimethyl sulfoxide | 10 | ml | 24 | hours | 50° C. | 2 | hours |
| | | | | 15 | minutes | | 10 | minutes |
| Example 38 | Ethanol | 25 | ml | 24 | hours | 50° C. | 2 | hours |

### Example 39

Suvorexant (500 mg) and methyl t-butyl ether (30 ml) were introduced in 100 ml round bottom flask. The heterogeneous mixture so obtained was slurried with stirring at about 55° C. for about 2 hours 10 minutes and the solid substance was then filtered under vacuum. The product was dried at 50° C. in air tray drier for 2 hours to obtain Form B.

### Example 40

Suvorexant (500 mg) was taken in n-propanol (25 ml) and heated at 80° C. to get clear solution. The resulting solution was cooled slowly to room temperature and then stirred for 2 hours. The resulting crystals were filtered and then dried at 50° C. for 1 hour to obtain Form B.

### Example 41-48

The process of example 40 was repeated wherein n-propanol was replaced by following solvents to obtain Form B:

| Example | Solvent | Quantity | |
|---|---|---|---|
| Example 41 | Methyl isobutyl | 12.5 | ml |
| | ketone | | |
| Example 42 | Acetonitrile | 15 | ml |
| Example 43 | 1-butanol | 10 | ml |
| Example 44 | 1-Pentanol | 15 | ml |
| Example 45 | Isopropyl acetate | 25 | ml |
| Example 46 | n-butyl acetate | 20 | ml |

### Example 47

Suvorexant (500 mg) was taken in isobutanol (20 ml) and heated at 70° C. to get clear solution. The resulting solution was cooled slowly to room temperature and then stirred for 24 hour. The resulting crystals were filtered and then dried to obtain in air tray drier at 50° C. for 2 hour to obtain Form B.

### Example 48

Suvorexant (500 mg) was taken in propyl acetate (10 ml) and heated at 70° C. to get clear solution. The resulting solution was filtered and the filtrate was cooled slowly to room temperature. The reaction mixture was stirred at room temperature for 5 hours. The resulting crystals were filtered and then dried in air tray drier at 50° C. for 2 hour to obtain Form B.

### Example 49

Suvorexant (500 mg) was taken in methyl acetate (15 ml) and heated to 65° C. to get clear solution. The resulting solution was cooled to 27° C. and stirred for 1 hour then further cooled to 0° C. and stirred at 0° C. for 2 hours. The precipitated product was filtered and then dried at 50° C. in air tray drier for 2 hours to obtain Form B.

### Example 50

Suvorexant (500 mg) was taken in Isoamyl alcohol (20 ml) and heated to 80° C. to get clear solution. The resulting solution was cooled to 27° C. and stirred for 1 hour then further cooled to 0° C. and stirred at 0° C. for 2 hours. The precipitated product was filtered and then dried at 50° C. in air tray drier for 2 hours to obtain Form B.

### Example 51

Suvorexant (500 mg) was taken in Isopropyl alcohol (25 ml) and heated to 80° C. to get clear solution. The resulting solution was cooled to 27° C. and stirred for 1 hour 10 minutes then further cooled to 0° C. and stirred at 0° C. for 50 minutes. The precipitated product was filtered and then dried at 50° C. in air tray drier for 2 hour to obtain Form B.

### Example 52

Suvorexant (500 mg) was taken in Methyl ethyl ketone (20 ml), heated to 78° C. to get clear solution and filtered. The resulting filtrate was cooled to 27° C. and stirred for 15 minutes then further cooled to 0° C. and stirred at 0° C. for 1 hour 30 minutes. The precipitated product was filtered and then dried at 50° C. in air tray drier for 2 hour 10 minutes to obtain Form B.

### Example 53

Suvorexant (500 mg) was dissolved under stirring at 65° C. in Dimethyl carbonate (25 ml). The resulting solution was slowly cooled to room temperature and n-heptane (50 ml) was charged in to the solution. After the addition of n-heptane to the solution was complete, resulting mixture was stirred for 5 hours 30 minutes. Precipitated crystals were collected by filtration and dried at 50° C. for 2 hours in air tray drier to obtain Form B.

### Example 54

Suvorexant (500 mg) was dissolved under stirring at 55° C. in acetone (30 ml) and the resulting solution was filtered. Filtrate was cooled to room temperature and added slowly to n-heptane (60 ml). After the addition of the solution of suvorexant to heptane was complete, resulting mixture was stirred for 21 hours 30 minutes. Precipitated crystals were collected by filtration and dried at 60° C. for 2 hours in air tray drier to obtain Form B.

### Example 55

Suvorexant (500 mg) was dissolved under stirring at 55° C. in Tetrahydrofuran (15 ml) and the resulting solution was filtered. Filtrate was cooled to room temperature and added slowly to n-heptane (45 ml). After the addition of the solution of suvorexant to heptane was complete, resulting mixture was stirred for 2 hours 30 minutes. Precipitated crystals were collected by filtration and dried at 50° C. for 2 hours 15 minutes in air tray drier to obtain Form B.

### Example 56

Suvorexant (500 mg) was dissolved under stirring at 60° C. in n-butanol (20 ml) and the resulting solution was filtered. Filtrate was cooled to room temperature and added slowly to n-heptane (80 ml). After the addition of the solution of suvorexant to heptane was complete, resulting mixture was stirred for 16 hours. Precipitated crystals were collected by filtration and dried at 60° C. for 1 hour 30 minutes in air tray drier to obtain Form B.

### Example 57

Suvorexant (500 mg) was dissolved under stirring at 60° C. in Dimethyl carbonate (25 ml) and the resulting solution was filtered. Filtrate was cooled to room temperature and added slowly to n-heptane (60 ml). After the addition of the solution of suvorexant to heptane was complete, resulting mixture was stirred for 1 hour. Precipitated crystals were collected by filtration and dried at 60° C. for 2 hours in air tray drier to obtain Form B.

### Example 58

Suvorexant (500 mg) was dissolved under stirring at 68° C. in Ethyl acetate (10 ml) and the resulting solution was filtered. Filtrate was cooled to room temperature and added slowly to n-heptane (30 ml). After the addition of the solution of suvorexant to heptane was complete, resulting mixture was stirred for 22 hours 30 minutes. Precipitated crystals were collected by filtration and dried at 50° C. for 2 hours 10 minutes in air tray drier to obtain Form B.

### Example 59

Suvorexant (500 mg) was dissolved under stirring at 80° C. in n-propanol (40 ml) and the resulted solution was filtered. Filtrate was cooled to room temperature and added slowly to water (120 ml). After the addition of the solution of suvorexant to water was complete, resulting mixture was stirred at 28° C. for 30 minutes further cooled to 0° C. and stirred at 0° C. for 24 hours. Precipitated crystals were collected by filtration and dried at 50° C. in air tray drier for 2 hour to obtain Form B.

### Example 60

Suvorexant (500 mg) was dissolved under stirring in THF (10 ml). Water (15 ml) was added slowly to the solution. After the addition of water to the solution of suvorexant was complete, resulting mixture was stirred at room temperature for 24 hours. Precipitated crystals were collected by filtration and dried at 50° C. in air tray drier to obtain Form B.

### Example 61

Suvorexant (500 mg) was dissolved under stirring at 80° C. in n-propanol (40 ml) and the resulted solution was filtered. Filtrate was cooled to room temperature and added slowly to petroleum ether (120 ml). After the addition of the solution of suvorexant to petroleum ether was complete, resulting mixture was stirred at 0° C. for 20 hours 30 minutes. Precipitated crystals were collected by filtration and dried at 50° C. for 2 hours in air tray drier to obtain Form B.

### Example 62

Suvorexant (500 mg) was dissolved under stirring at 60° C. in Isopropyl alcohol (40 ml). The resulting solution was cooled to room temperature and added slowly to petroleum ether (120 ml). After the addition of the solution of suvorexant to petroleum ether was complete, resulting mixture was stirred till isolation of solid. Precipitated crystals were collected by filtration and dried at 50° C. in air tray drier to obtain Form B.

### Example 63

Suvorexant (4 gm) was taken in methanol (160 ml) and heated at 70° C. to get solution. The resulting solution was filtered to get clear solution, slowly cooled to 20° C. and then stirred for 30 minute. Precipitated crystals were collected by filtration under vacuum. The crystals were subjected to air tray drying for 30 minute at 50° C. to obtain Form C.

### Example 64

Suvorexant (500 mg) was taken in methanol (20 ml) and heated at 70° C. to get solution. The resulting solution was filtered to get clear solution, slowly cooled to 20° C. and then stirred for 30 minute. Precipitated crystals were collected by filtration under vacuum. The crystals were subjected to air tray drying for 1 hour at 70° C.

Further the crystals were dried in vacuum tray drier at 75° C. for 6.5 hour then temperature was raised to 90° C. and dried for 4 hour to obtain Form D.

### Example 65

Suvorexant (500 mg) and methanol (50 ml) were introduced in round bottom flask, heated to 60° C. to get clear solution and the resulted solution was filtered. Methanol was evaporated from the filtrate under vacuum at 60° C. under vacuum. To the resulted gummy residue ethyl acetate (2.5 ml) and hexane (5 ml) was added and stirred at 28° C. for 24 hours. The solid product was filtered and dried in vacuum to obtain Form E.

### Example 66

Suvorexant (500 mg, HPLC purity 98.69%) and acetonitrile (22 ml) were introduced in round bottom flask, heated to 70° C. to get clear solution and the resultant solution was filtered. Acetonitrile was evaporated from the filtrate at 70° C. under vacuum. To the resulted gummy residue ethyl acetate (2.5 ml) and hexane (5 ml) was added and stirred at 28° C. for 24 hours. The solid product was filtered and dried in vacuum to obtain Form E.

(HPLC Purity 99.92%).

### Example 67

Suvorexant (500 mg) and acetone (25 ml) were introduced in round bottom flask, heated to 55° C. to get clear solution and the resulted solution was filtered. Acetone was evaporated from the filtrate under vacuum at 55° C. under vacuum. To the resulted gummy residue ethyl acetate (2.5 ml) and hexane (5 ml) was added and stirred at 28° C. for 24 hours. The solid product was filtered and dried in vacuum to obtain Form E.

### Example 68

Suvorexant (5 gm) and methanol (200 ml) were introduced in round bottom flask, heated to 60° C. to get clear solution and the resulted solution was filtered. Filtrate was allowed to cool at room temperature and spray dried by a spray drying machine under the conditions of aspirator: 70%, feed rate: 20%, inlet temperature: 70° C., atomization pressure: 5 kg/cm² to obtain amorphous Suvorexant.

### Example 69

Suvorexant (500 mg) was dissolved under stirring at 80° C. in formamide (50 ml) and the resulted solution was filtered. Filtrate was cooled to room temperature and added slowly to water (150 ml). After the addition of the solution of suvorexant to water was complete, resulting mixture was stirred for 5 minutes. Precipitate crystals were collected by filtration under vacuum and dried in air tray dried at 60° C. for 1 hour 40 minutes to obtain amorphous suvorexant.

### Example 70

Suvorexant (500 mg) was dissolved under stirring at 60° C. in Isopropyl alcohol (40 ml) and the resulted solution was filtered. Filtrate was cooled to room temperature and added slowly to water (120 ml). After the addition of the solution of suvorexant to water was complete, resulting mixture was stirred at 28° C. for 30 minutes. Precipitated crystals were collected by filtration and dried to obtain amorphous suvorexant.

### Example 71

Suvorexant (500 mg) and methanol (30 mL) were charged in to a round-bottom flask at 25-35° C. The contents were heated to 60-70° C. and stirred to dissolve suvorexant completely. The resulting solution was filtered, and the filtrate evaporated completely in Büchi® Rotavapor® at 55-60° C. under reduced pressure to afford 450 mg of amorphous suvorexant.

PXRD Pattern is shown as FIG. 7.

### Example 72

Suvorexant (500 mg) and acetonitrile (30 ml) were charged in to a round-bottom flask at 25-35° C. The contents were heated to 60-70° C. and stirred to dissolve suvorexant completely. The resulting solution was filtered, and the filtrate evaporated completely in Büchi® Rotavapor® at 55-60° C. under reduced pressure to afford 440 mg of amorphous suvorexant.

### Example 73

Suvorexant (500 mg) and acetone (30 mL) were charged in to a round-bottom flask at 25-35° C. The contents were heated to 55-65° C. and stirred to dissolve suvorexant completely. The resulting solution was filtered, and the filtrate evaporated completely in Büchi® Rotavapor® at 55-60° C. under reduced pressure to afford 410 mg of amorphous suvorexant.

### Example 74

Suvorexant (500 mg) and N,N-dimethylacetamide (4 mL) were charged in to a round-bottom flask at 25-35° C. The contents were heated to 80° C. and stirred to dissolve suvorexant completely. The resulting solution cooled to 25-35° C. and stirred for 1 hour. The reaction mass further cooled to 0° C. and stirred for 14-16 hours. Water (25 mL) was charged at once to the above solution at 25-30° C. and stirred for 30 minutes. The obtained solid was collected by filtration and dried at 50° C. to afford 320 mg of amorphous suvorexant.

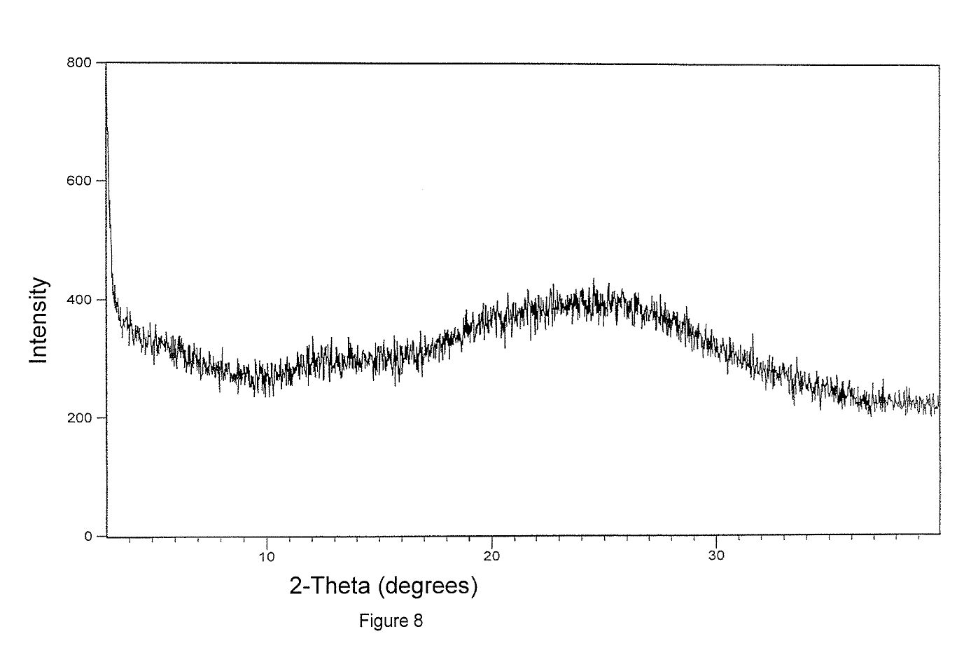

PXRD Pattern is shown as FIG. 8.

### Example 75

Suvorexant (1.0 g), Povidone K-30 (1.0 g) and methanol (450 mL) were charged in to a round bottom flask at 25-35° C. The contents were heated to 55-60° C. and stirred to dissolve the contents completely. The resulting solution was filtered and the filtrate evaporated completely under reduced pressure at 55-60° C. under reduced pressure to afford 1.78 g of a solid dispersion of amorphous suvorexant with povidone.

PXRD Pattern is shown as FIG. 9.

### Example 76

Suvorexant (500 mg) was dissolved under stirring at 60° C. in isopropyl alcohol (40 ml). The resulting solution was cooled to room temperature and added slowly to water (120 ml). After the addition of the solution of suvorexant to water was complete, resulting mixture was stirred till isolation of solid. The obtained solid is collected by filtration and dried under vacuum to afford 350 mg of amorphous suvorexant.

### Example 77

Suvorexant (500 mg) was taken in ethyl acetate (14 ml) and heated at 65° C. to get clear solution. The resulting solution was evaporated under vacuum at 65° C. to obtain 480 mg of amorphous suvorexant.

### Example 78

Suvorexant (10 gm) was dissolved under stirring at 80° C. in isopropyl alcohol (500 ml). The resulting solution was cooled to room temperature and added slowly to n-heptane (1500 ml). After the addition of the solution of suvorexant to n-heptane was complete, resulting mixture was stirred till isolation of solid. Precipitated crystals were collected by filtration and dried in vacuum tray drier at 60° C. to obtain Form B (4.1 gm).

### Example 79

Suvorexant (500 mg) was dissolved under stirring at 65-70° C. in methanol (20 ml). Toluene (50 ml) was added slowly to the solution. After the addition of toluene to the solution of suvorexant was complete, resulting mixture was stirred at 70° C. for 5 hours. The solution was subjected to vacuum distillation at 78° C. to obtain 430 mg of Form B.

### Example 80

Suvorexant (500 mg) and n-heptane (5 ml) were introduced in 100 ml round bottom flask. The heterogeneous mixture so obtained was slurried with stirring at about 60° C. for about 1 hour and the solid substance was then filtered under vacuum to obtain 450 mg of Form B.

### Example 81

Suvorexant (1 gm) was taken in methanol (40 ml) and heated at 50° C. to get clear solution. The resulting solution was filtered to get clear solution, slowly cooled to room temperature and then stirred for 3 hours. Precipitated crystals were collected by filtration under vacuum. The crystals were subjected to air tray drying for 2 hours at 50° C. to obtain Form C. The obtained Form C and toluene (80 ml) were introduced in round bottom flask. The heterogeneous mixture so obtained was slurried with stirring at about 78° C. for about 1 hour and the solid substance was then filtered under vacuum to obtain 805 mg of Form B.

### Example 82

Suvorexant Form B (500 mg) and water (10 ml) were introduced in round bottom flask. The heterogeneous mixture so obtained was slurried with stirring at about 80° C. for about 1 hour and the solid substance was then filtered under vacuum at 80° C. The filtered solid was analyzed after 1 hour and 4 hours. The solid obtained after 1 hour and 4 hours were Form B up on analysis with XRD.

### Example 83

Suvorexant Form B (500 mg) and water (10 ml) were introduced in round bottom flask. The heterogeneous mixture so obtained was slurried with stirring at about room temperature for about 1 hour. The heterogeneous mixture was divided in to two parts. The first part was filtered under vacuum. The filtered solid was analyzed after 2 hour and 4 hours. The solid obtained after 2 hour and 4 hours were Form B up on analysis with XRD. The second part was stirred over night at room temperature and then filtered under vacuum. The solid obtained from second part was Form B up on analysis with XRD.

### Example 84

Suvorexant Form D (1.5 gm) and water (30 ml) were introduced in round bottom flask. The heterogeneous mixture so obtained was slurried with stirring over night at about room temperature and the solid substance was then filtered under vacuum to obtain Form G.

### Example 85

Suvorexant (1 gm) was taken in ethanol (50 ml) and heated at 65° C. to get solution. The resulting solution was filtered to get clear solution, slowly cooled to 0-5° C., purged with nitrogen and stirred for 2 hours. Precipitated crystals were collected by filtration under vacuum. The obtained crystals were Form H. The crystals were further subjected to air tray drying for 60 minute at 50° C. to obtain mixture of Form H and Form D. The temperature of air tray drier was raised to 70° C. and crystals were further dried for 1 hour to obtain Form D.

### Example 86

Suvorexant Form D (500 mg) and water (15 ml) were introduced in round bottom flask. The heterogeneous mixture so obtained was slurried with stirring for 5 hours at about 80° C. and the solid substance was then filtered under vacuum to obtain Form A.

### Example 87

Suvorexant Form C (500 mg) and water (10 ml) were introduced in round bottom flask. The heterogeneous mixture so obtained was slurried with stirring for 5 hours at about 80° C. and the solid substance was then filtered under vacuum to obtain Form A.

### Example 88

Suvorexant (1 gm) and MTBE (30 ml) were introduced in round bottom flask. The heterogeneous mixture so obtained was slurried with stirring for 1 hour at about 55° C. and the solid substance was then filtered under vacuum to obtain Form A.

### Example 89

Suvorexant (500 mg) was dissolved under stirring at 55° C. in methanol (15 ml). Water (45 ml) was charged in to the solution. After the addition of water to the solution was complete, resulting mixture was stirred for 5 hours at 55° C. The mixture was cooled to room temperature and further stirred for 3 hours. Precipitated crystals were collected by filtration under vacuum to obtain 210 mg of Form A.

### Example 90

Suvorexant (300 mg) was dissolved under stirring at 55° C. in acetone (15 ml). MTBE (50 ml) was charged in to the solution. After the addition of MTBE to the solution was complete, resulting mixture was stirred for 5 hours at 55° C. Precipitated crystals were collected by filtration under vacuum to obtain 250 mg of Form A.

### Example 91

Suvorexant (150 mg) and heptane (4 ml) were introduced in 100 ml round bottom flask. The heterogeneous mixture so obtained was slurried with stirring at about 70° C. for about 20 hours and the solid substance was filtered under vacuum. The product was dried in air tray drier at 50° C. for 1 hour to obtain Form A.

### Example 92

Suvorexant (150 mg) and water (4 ml) were introduced in 100 ml round bottom flask. The heterogeneous mixture so obtained was slurried with stirring at about 70° C. for about 20 hours and the solid substance was filtered under vacuum. The product was dried in air tray drier at 50° C. for 1 hour to obtain Form A.

### Example 93

Suvorexant (150 mg) and ethyl acetate (4 ml) were introduced in 100 ml round bottom flask. The heterogeneous mixture so obtained was slurried with stirring at about 70° C. for about 20 hours and the solid substance was filtered under vacuum. The product was dried in air tray drier at 50° C. for 1 hour to obtain Form A.

### Example 94

Suvorexant (150 mg) and heptane (4 ml) were introduced in 100 ml round bottom flask. The heterogeneous mixture so obtained was slurried with stirring at about 70° C. for about 5 hours. The mixture was cooled to −10° C. over a period of 2 hours and maintained for 12 hours. The solid filtered under vacuum and dried in air tray drier at 50° C. for 1 hour to obtain Form A.

### Example 95

Suvorexant (150 mg) and water (4 ml) were introduced in 100 ml round bottom flask. The heterogeneous mixture so obtained was slurried with stirring at about 70° C. for about 5 hours. The mixture was cooled to −10° C. over a period of 2 hours and maintained for 12 hours. The solid filtered under vacuum and dried in air tray drier at 50° C. for 1 hour to obtain Form A.

### Example 96

Suvorexant (150 mg) and ethyl acetate (4 ml) were introduced in 100 ml round bottom flask. The heterogeneous mixture so obtained was slurried with stirring at about 70° C. for about 5 hours. The mixture was cooled to −10° C. over a period of 2 hours and maintained for 12 hours. The solid filtered under vacuum and dried in air tray drier at 50° C. for 1 hour to obtain Form A.

1.-43. (canceled)

44. A crystalline Form A of suvorexant.

45. The crystalline Form A of suvorexant according to claim 44, having an X-ray powder diffraction pattern comprising peaks at 2θ values of about 12.07°, 14.11°, 22.16° and 25.45°±0.2°.

46. The crystalline Form A of suvorexant according to claim 44, further comprising peaks at 2θ values of about 18.53° and 19.94°±0.2°.

47. The crystalline Form A of suvorexant according to claim 44, further comprising peaks at 2θ values of about 25.97° and 26.75°±0.2°.

48. The crystalline Form A of suvorexant according to claim 44, having an X-ray powder diffraction pattern with peaks located substantially in accordance with FIG. 1.

49. A pharmaceutical composition comprising the crystalline Form A of suvorexant according to claim 44 and a pharmaceutically acceptable carrier.

50. A crystalline Form B of suvorexant.

51. The crystalline Form B of suvorexant according to claim 50, having an X-ray powder diffraction pattern comprising peaks at 2θ values of about 19.93°, 26.73° and 31.38°±0.2°.

52. The crystalline Form B of suvorexant according to claim 50, further comprising peaks at 2θ values of about 12.06°, 15.38°, 25.43° and 25.94°±0.2°.

53. The crystalline Form B of suvorexant according to claim 50, further comprising peaks at 2θ values of about 18.53°, 22.16° and 23.21°±0.2°.

54. The crystalline Form B of suvorexant according to claim 50, having an X-ray powder diffraction pattern with peaks located substantially in accordance with FIG. 2.

55. A pharmaceutical composition comprising the crystalline Form B of suvorexant according to claim 50 and a pharmaceutically acceptable carrier.

56. A crystalline Form C of suvorexant.

57. The crystalline Form C of suvorexant according to claim 56, having an X-ray powder diffraction pattern comprising peaks at 2θ values of about 9.70°, 22.20° and 23.91°±0.2°.

58. The crystalline Form C of suvorexant according to claim 56, further comprising peaks at 2θ values of about 11.66°, 12.72° and 20.31°±0.2°.

59. The crystalline Form C of suvorexant according to claim 56; further comprising peaks at 2θ values of about 14.16° and 26.47°±0.2°.

60. The crystalline Form C of suvorexant according to claim 56, having an X-ray powder diffraction pattern with peaks located substantially in accordance with FIG. 3.

61. A pharmaceutical composition comprising the crystalline Form C of suvorexant according to claim 56 and a pharmaceutically acceptable carrier.

62. A crystalline Form D of suvorexant.

63. The crystalline Form D of suvorexant according to claim 62, having an X-ray powder diffraction pattern comprising peaks at 2θ values of about 4.26° and 12.66°±0.2°.

64. The crystalline Form D of suvorexant according to claim 62, further comprising peaks at 2θ values of about 20.59° and 23.22°±0.2°.

65. The crystalline Form D of suvorexant according to claim 62, further comprising peaks at 2θ values of about 19.01°, 25.62° and 28.51°±0.2°.

66. The crystalline Form D of suvorexant according to claim 62 having an X-ray powder diffraction pattern with peaks located substantially in accordance with FIG. 4.

67. A pharmaceutical composition comprising the crystalline Form D of suvorexant according to claim 62 and a pharmaceutically acceptable carrier.

68. A crystalline Form E of suvorexant.

69. The crystalline Form E of suvorexant according to claim 68, having an X-ray powder diffraction pattern comprising peaks at 2θ values of about 10.53 and 16.05°±0.2°.

70. The crystalline Form E of suvorexant according to claim 68, further comprising peaks at 2θ values of about 14.10°, 17.49° and 21.05°±0.2°.

71. The crystalline Form E of suvorexant according to claim 68, having an X-ray powder diffraction pattern with peaks located substantially in accordance with FIG. 5.

72. A pharmaceutical composition comprising the crystalline Form E of suvorexant according to claim 68 and a pharmaceutically acceptable carrier.

73. A crystalline Form G of suvorexant.

74. The crystalline Form G of suvorexant according to claim 73, having an X-ray powder diffraction pattern comprising peaks at 2θ values of about 8.14°, 14.97°, 17.92°, 20.25° and 21.74°±0.2°.

75. The crystalline Form G of suvorexant according to claim 73, further comprising peaks at 2θ values of about 4.12°, 9.64°, 14.07°, 22.15°, 23.09° and 23.81°±0.2°.

76. The crystalline Form G of suvorexant according to claim 73, having an X-ray powder diffraction pattern with peaks located substantially in accordance with FIG. 10.

77. A pharmaceutical composition comprising the crystalline Form G of suvorexant according to claim 73 and a pharmaceutically acceptable carrier.

78. A crystalline Form H of suvorexant.

79. The crystalline Form H of suvorexant according to claim 78, having an X-ray powder diffraction pattern comprising peaks at 2θ values of about 7.33°, 9.08°, 11.02°, 14.71°, 22.74° and 29.20°±0.2°.

80. The crystalline Form H of suvorexant according to claim 78, further comprising peaks at 2θ values of about 12.22°, 12.89°, 18.41°, 19.02°, 20.27°, 23.08°, 23.73°, and 26.30°±0.2°.

81. The crystalline Form H of suvorexant according to claim 78, having an X-ray powder diffraction pattern with peaks located substantially in accordance with FIG. 11.

82. A pharmaceutical composition comprising the crystalline Form H of suvorexant according to claim 78 and a pharmaceutically acceptable carrier.

83. Amorphous suvorexant.

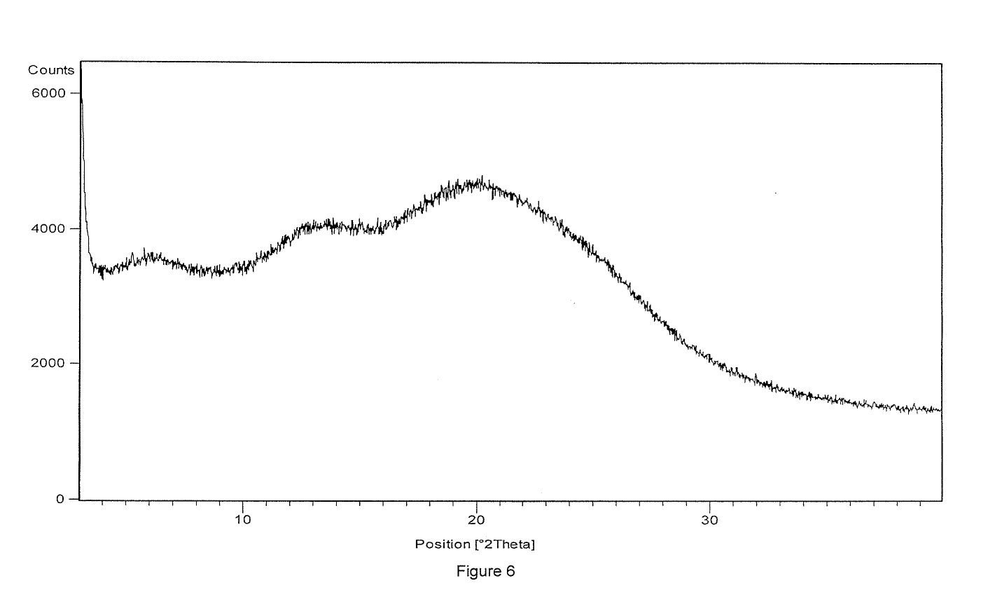

84. The amorphous suvorexant according to claim 83, substantially in accordance with FIG. 6.

85. A pharmaceutical composition comprising the amorphous suvorexant according to claim 83 and a pharmaceutically acceptable carrier.

86. A solid dispersion of suvorexant comprising of suvorexant and pharmaceutically acceptable carrier.

87. An amorphous solid dispersion of suvorexant comprising suvorexant and pharmaceutically acceptable carrier.

88. The solid dispersion of suvorexant according to claim 86, comprising suvorexant and povidone.

89. A pharmaceutical composition comprising the solid dispersion of suvorexant according to claim 86 together with one or more pharmaceutically acceptable carriers or excipients.

90. A pharmaceutical composition comprising the amorphous solid dispersion of suvorexant according to claim 87 together with one or more pharmaceutically acceptable carriers or excipients.

91. The amorphous solid dispersion of suvorexant according to claim 87, substantially in accordance with FIG. 9.

92. A process for preparing a solid dispersion of suvorexant according to claim 86 together with one or more pharmaceutically acceptable carriers, comprising:
a) providing a solution or suspension of suvorexant in combination with one or more pharmaceutically acceptable carriers in a solvent or mixture of solvents; and
b) isolating solid dispersion of suvorexant together with one or more pharmaceutically acceptable carriers.

93. A process for preparing an amorphous solid dispersion of suvorexant according to claim 87 together with one or more pharmaceutically acceptable carriers, comprising:
a) providing a solution or suspension of suvorexant in combination with one or more pharmaceutically acceptable carriers in a solvent or mixture of solvents; and
b) isolating amorphous solid dispersion of suvorexant together with one or more pharmaceutically acceptable carriers.
